(19) United States
(12) Patent Application Publication
(10) Pub. No.: US 2013/0239803 A1
(43) Pub. Date: Sep. 19, 2013

(54) System and Method For Air Replacement and Positive Air Pressure Isolation

(71) Applicant: AMERICAN INNOVATIVE RESEARCH CORP., Andover, MA (US)

(72) Inventor: David W. Palmer, Andover, MA (US)

(73) Assignee: AMERICAN INNOVATIVE RESEARCH CORP., Andover, MA (US)

(21) Appl. No.: 13/780,546

(22) Filed: Feb. 28, 2013

Related U.S. Application Data
(63) Continuation-in-part of application No. 13/306,489, filed on Nov. 29, 2011, now Pat. No. 8,485,874.
Application No. 13/306,489 is a continuation of application No. 12/603,107, filed on Oct. 21, 2009, now Pat. No. 8,087,980.
Application No. 12/603,107 is a continuation of application No. 11/805,776, filed on May 24, 2007, now Pat. No. 7,625,277.
(60) Provisional application No. 60/802,977, filed on May 24, 2006.
(60) Provisional application No. 61/605,390, filed on Mar. 1, 2012.

(51) Int. Cl. A61L 9/20

(52) U.S. Cl. CPC A61L 9/20
USPC 95/22; 422/4; 250/432 R; 340/611; 62/3.2; 454/239; 96/224

(57) ABSTRACT

A method for reducing airborne contamination within a home includes installing an air-pressure control system within a window of the home, and drawing air from outside the home through the system inlet and an airflow path through the system. As it passes through the airflow path, a germicidal radiation chamber with a UV light source sterilizes the air. Additionally, filter(s) may clean the drawn air by removing particulates from the air as it passes through the filter. The method may then introduce the sterilized/cleaned air into the home through the system outlet. The sterilized/cleaned air may displace an equal volume of contaminated air within the home.

### RELATED APPLICATIONS

This application is a continuation in part of co-pending U.S. application Ser. No. 13/306,489, entitled “Positive Air Pressure Isolation System,” filed Nov. 29, 2011, assigned attorney docket number 3116/104, and naming David W. Palmer as inventor the disclosure of which is incorporated herein, in its entirety, by reference.

As a consequence of this priority claim, this patent also claims priority from the following:

U.S. application Ser. No. 12/603,107, entitled “Positive Air Pressure Isolation System,” filed Oct. 21, 2009, assigned attorney docket number 3116/103, and naming David W. Palmer as inventor, the disclosure of which is incorporated herein, in its entirety, by reference;

U.S. application Ser. No. 11/805,776, entitled “Positive Air Pressure Isolation System,” filed May 24, 2007, assigned attorney docket number 3116/102, and naming David W. Palmer as inventor, the disclosure of which is incorporated herein, in its entirety, by reference;

U.S. provisional application Ser. No. 60/802,977, filed May 24, 2006, entitled “Positive Air Pressure Isolation System,” assigned attorney docket number 3116/101, and naming David W. Palmer as inventor, the disclosure of which is hereby incorporated by reference in its entirety.

Additionally, the present application also claims priority from U.S. provisional application Ser. No. 61/605,390, filed Mar. 1, 2012, entitled “System and Method for Air Replacement,” assigned attorney docket number 3116/105, and naming David W. Palmer as inventor, the disclosure of which is hereby incorporated by reference in its entirety.

### FIELD OF THE INVENTION

The invention generally relates to improving the air quality within a home and, more particularly, to the management and cleaning of air flow in or out of a closed space to displace contaminated air and/or produce a constant positive or negative room air pressure.

### SUMMARY OF THE INVENTION

In accordance with one embodiment of the present invention, a method for reducing airborne contamination within a home may include installing an air-pressure control system within a window of the home, and drawing air from outside the home. The air-pressure control system may include a system inlet, a system outlet, a variable speed fan configured to operate at a speed, and a motor controller in communication with the fan and configured to control the speed of the fan. The system may also include a solid state anemometer configured to monitor an air pressure differential between the system inlet and the system outlet, a closed-loop controller in communication with the motor controller and the solid state anemometer, and a germicidal radiation chamber. The closed-loop controller is configured to vary the speed of the fan based on the pressure differential between the inlet and outlet of the system. The germicidal radiation chamber may be located within an airflow path in the air-pressure control system, and may include at least one UV light source.

As the air is drawn from outside the home, it may be drawn through the system inlet and the airflow path, and the germicidal radiation chamber may sterilize the air as it passes through the airflow path. The method may then introduce (e.g., at a flowrate between 20 cubic feet per minute and 75 cubic feet per minute) the sterilized air into the home through the system outlet. The sterilized air may displace an equal volume of contaminated air within the home.

In accordance with some embodiments, the air-pressure control system may include at least one filter located within the airflow path. The filter may clean the drawn air by removing particulates from the drawn air as it passes through the filter. The filter may be located at a first end of the germicidal radiation chamber. The air-pressure control system further may also include a second filter located at a second end of the germicidal radiation chamber.

The air-pressure control system (e.g., via the germicidal radiation chamber and filter) may remove volatile organic compounds from the drawn air, and the introduced air may contain substantially no particles (e.g., dust mites, animal dander, bacteria, lead paint, household dust, cooking smoke and grease, wood and tobacco smoke, and smog) between 5.0 microns and 0.3 microns. Additionally, displacing the contaminated air within the home may improve the air quality within the home. The contaminated air may include volatile organic compounds, airborne micro-contamination, harmful gases, dust mites, animal dander, bacteria, lead paint, household dust, cooking smoke and grease, wood and tobacco smoke, and/or smog.

In some embodiments, the air-pressure control system may include an adjustable frame that, in turn, includes a top rail and a first and second adjustable side rail. Installing the air pressure control system within the window may include (1) inserting the air-pressure control system into the window such that the adjustable frame is within the window frame, (2) and adjusting the first and second side rails to expand the adjustable frame to fit the window frame. The adjustable frame may include a tape measure having a zero point located on a center point of the top rail.

The tape measure may include a first set of increasing numbers and a second set of increasing numbers. The first set of increasing numbers may increase to the left of the zero point, and the second set of increasing numbers may increase to the right of the zero point. When the first and second side rails are adjusted to expand the adjustable frame, the numbers from the first and second set of increasing numbers may be exposed. Installing the system may also include aligning the zero point with a center of the window frame, and adjusting first and second side rails such that the exposed numbers from the first and second set of increasing numbers match.

In further embodiments, the method may include providing a first and second cover for the adjustable frame, cutting the first and second covers based upon the exposed numbers from the first and second set of increasing numbers, and installing the first and second covers into the adjustable frame. The first cover may cover a first space between a side of the air-pressure control system and the first side rail. The second cover may cover a second space between an opposing side of the air-pressure control system and the second side rail.

The air-pressure control system may also include a thermostat and a thermoelectric device configured to adjust the temperature of the air drawn into the air-pressure control system. Additionally or alternatively, the air-pressure control system may include a thermocouple located within the germicidal radiation chamber and a heater located upstream of the germicidal radiation chamber. The thermocouple may be connected to the closed-loop controller and may be configured to measure a temperature of the air passing through the germicidal radiation chamber. The closed-loop controller may adjust the power to the main heater based upon the measured temperature to heat the air passing through the air-pressure control system.

In some embodiments, the air-pressure control system may also include a humidistat located between the system inlet and the system outlet, and connected to the thermoelectric device. The humidistat may be configured to measure the humidity of the drawn air. The thermoelectric device may be configured to dehumidify the drawn air based upon the measured humidity.

In accordance with further embodiments, a system for reducing airborne contamination within a home may include a housing defining the structure of the system, and an adjustable frame extending around the housing. The housing may be configured to fit within a window of the home, and the adjustable frame may be configured to expand to at least one dimension of the window. The system may also include a system inlet, a system outlet, a variable-speed fan configured to operate at a speed, and a motor controller in communication with the fan and configured to control the speed of the fan.

Some embodiments of the system may also include a solid state anemometer, a closed-loop controller, and a germicidal radiation chamber. The solid state anemometer may be configured to monitor an air pressure differential between the system inlet and the system outlet. The closed-loop controller may be in communication with the motor controller and the solid state anemometer, and may be configured to vary the speed of the fan based on the pressure differential between the inlet and outlet of the system. The germicidal radiation chamber may be located within an airflow path in the system, and may include at least one UV light source. The germicidal radiation chamber may be configured to sterilize the air as it passes through the airflow path. The sterilized air may displace an equal volume of contaminated air within the home.

The system may include at least one filter located within the airflow path (e.g., at a first end of the germicidal radiation chamber) that cleans the drawn air by removing particulates from the drawn air as it passes through the filter. Additionally or alternatively, the system may also include a second filter located at a second end of the germicidal radiation chamber. The system may be configured to introduce the sterilized air into the home at a flowrate between 20 cubic feet per minute and 75 cubic feet per minute, and/or may remove volatile organic compounds from the drawn air. The air exiting the system may contain substantially no particles (e.g., dust mites, animal dander, bacteria, lead paint, household dust, cooking smoke and grease, wood and tobacco smoke, and smog) between 5.0 microns and 0.3 microns. The sterilized air displacing the contaminated air within the home may improve the air quality within the home. The contaminated air may include volatile organic compounds, airborne micro-contamination, harmful gases, dust mites, animal dander, bacteria, lead paint, household dust, cooking smoke and grease, wood and tobacco smoke, and/or smog.

In some embodiments, the adjustable frame may include a top rail extending along a top surface of the housing, a first adjustable side rail located on a first side of the housing, and a second adjustable side rail located on a second side of the housing. The first and second adjustable rails may be configured to expand outwardly from the system such that the adjustable frame fits the window frame. The adjustable frame may include a tape measure having a zero point located on a center point of the top rail, a first set of increasing numbers, and a second set of increasing numbers. The first set of increasing numbers may increase to the left of the zero point, and the second set of increasing numbers may increase to the right of the zero point. The numbers from the first and second set of increasing numbers may be exposed as the first and second adjustable rails are expanded outwardly. When the system is installed in the window, the zero point may be aligned with a center of the window frame.

The adjustable frame may also include a first cover configured to cover a space between the first side of the housing and the first adjustable side rail, and a second cover configured to cover a space between the second side of the housing and the second adjustable side rail. The first and second covers may be sized based on the exposed numbers on the tape measure.

In further embodiments, the system may include a thermostat and a thermoelectric device configured to adjust the temperature of the air drawn into the system based upon a signal from the thermostat. Additionally or alternatively, the system may include a heater located upstream of the germicidal radiation chamber, and a thermocouple located within the germicidal radiation chamber. The thermocouple may be connected to the closed-loop controller and may be configured to measure the temperature of the air passing through the germicidal radiation chamber. The closed-loop controller may adjust the power to the main heater based upon the measured temperature to heat the air passing through the air-pressure control system. The system may also include a humidistat that may be located between the system inlet and the system outlet, and may be configured to measure the humidity of the drawn air. The thermoelectric device may be connected to the humidistat and may be configured to dehumidify the drawn air based upon the measured humidity.

In still further embodiments, the airflow path may be blackened to prevent UV reflection through the system inlet and system outlet. The closed-loop controller may include a microprocessor configured to compare an output from the solid state anemometer and a setpoint value, and adjust the speed of the fan based on the difference between the solid state anemometer output and the setpoint value. The system may also include a safety sensor in communication with the microprocessor. The microprocessor may alarm when the system is not operating at the setpoint values. Upon a change in condition within the home, the closed-loop controller may bring the fan to full speed, and then reduce the speed of the fan to obtain a setpoint value.

### BRIEF DESCRIPTION OF THE DRAWINGS

FIG. 1 shows an air-pressure-control system in accordance with an embodiment of the present invention.

FIG. 2 shows an airflow diagram of the system shown in FIG. 1.

FIG. 3 shows a logic diagram of the system shown in FIG. 1.

FIG. 4 shows an exemplary germicidal radiation chamber and electrical chassis in accordance with embodiments of the present invention.

FIG. 5 shows the exemplary germicidal radiation chamber of FIG. 4 in accordance with embodiments of the present invention.

FIG. 6 shows the germicidal radiation chamber of FIG. 4 with a chamber cover and UVC sensor in accordance with embodiments of the present invention.

FIG. 7 shows the inside of the germicidal radiation chamber of FIG. 4 in accordance with embodiments of the present invention.

FIG. 8 shows the inside of the electrical chassis of FIG. 4 in accordance with embodiments of the present invention.

FIG. 9 shows another view of the internals of the exemplary electrical chassis shown in FIG. 4 in accordance with embodiments of the present invention.

FIG. 10 shows a fan assembly with pre-filter in accordance with an embodiment of the air-pressure control system.

FIG. 11 shows an exemplary control panel in accordance with embodiments of the present invention.

FIG. 12 shows an exemplary outside shell with insulation on exposed elements in accordance with embodiments of the present invention.

FIG. 13 shows an alternative embodiment of an air-pressure-control system in accordance with an embodiment of the present invention.

FIG. 14 shows an airflow diagram of the system shown in FIG. 13.

FIG. 15 shows a logic diagram of the system shown in FIG. 13.

FIG. 16 shows an exemplary electrical schematic of the air-pressure-control system shown in FIG. 13, in accordance with embodiments of the present invention.

FIG. 17 shows an exemplary germicidal radiation chamber and electrical chassis of the system shown in FIG. 13, in accordance with embodiments of the present invention.

FIG. 18 shows the air-pressure-control system shown in FIG. 13 with an adjustable frame and mounting components, in accordance with embodiments of the present invention.

FIG. 19 shows the air-pressure-control system shown in FIG. 13 with the front panel removed to show the internal filters, in accordance with embodiments of the present invention.

FIG. 20 shows the air-pressure-control system shown in FIG. 13 with the back cover removed, in accordance with embodiments of the present invention.

FIG. 21 shows another view of the internals of the air-pressure-control system shown in FIG. 13 in accordance with embodiments of the present invention.

FIG. 22 shows an additional view of the internals of the air-pressure-control system shown in FIG. 13 in accordance with embodiments of the present invention.

FIG. 23 shows a further view of the internals of the air-pressure-control system shown in FIG. 13 in accordance with embodiments of the present invention.

FIG. 24 shows a flowchart showing the steps of one method for improving the air quality within a home, in accordance with some embodiments of the present invention.

FIG. 25 shows a further view of the internals of the air-pressure-control system shown in FIG. 13 including an air-flow path through the system, in accordance with embodiments of the present invention.

FIG. 26 shows a home and the progression of clean air through the home and the displacement of contaminated air, in accordance with some embodiments of the present invention.

FIG. 27 is a chart showing the type and size typical airborne contamination within a household.

### DETAILED DESCRIPTION OF SPECIFIC EMBODIMENTS

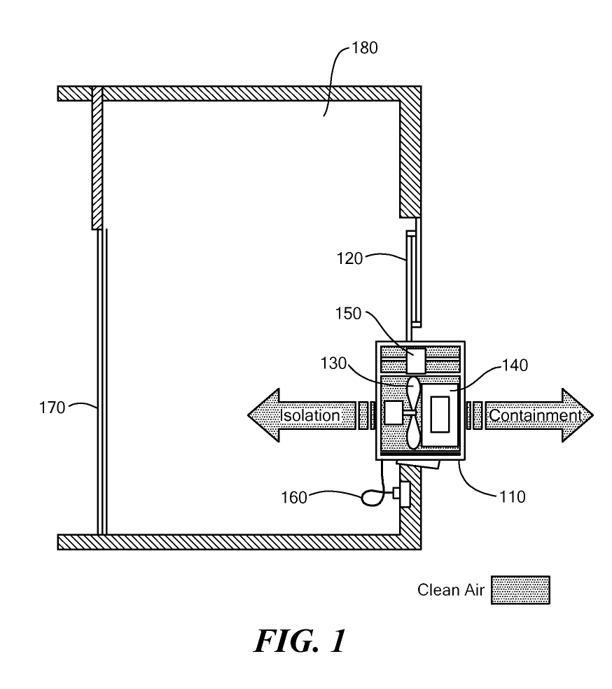

FIG. 1 shows an air-pressure-isolation system 110 in accordance with the present invention. The system 110 may be a through window, “plug and play” type system. As such, the system 110 can transform a closed space 180 into either an isolation or a containment room by placing the system 110 into a window 120 and plugging a power cord 160 into a standard wall socket. The inward facing side of the system 110 may have a stylish design so that it does not negatively impact the aesthetics of the closed space 180. The outward facing side of the system 110 may have a design that is suitable for exposure to the environment.

In an isolation configuration, a variable speed fan 130 forces clean air into the closed space 180, resulting in a positive pressure within the closed space 180. In order to produce a constant positive pressure consistent with surgical sites and clean rooms, the system 110 may control the air flow into the room, by varying the speed of the fan, to match the air flow out of the room through gaps around windows and doors. In the containment configuration, a variable-speed fan 130 forces air out of closed space 180, resulting in a negative room air pressure. In either orientation, a germicidal radiation chamber 140, located within a closed airflow path, cleans the air as it passes through system 110. If the system 110 is not installed in a window, the user can add an extension to the air path out of the germicidal radiation chamber 140 to reach the outside environment.

In some embodiments, the system 110 may contain multiple variable-speed fans. If more than one variable-speed fan is present, the fans may operate such that they force air in multiple directions.

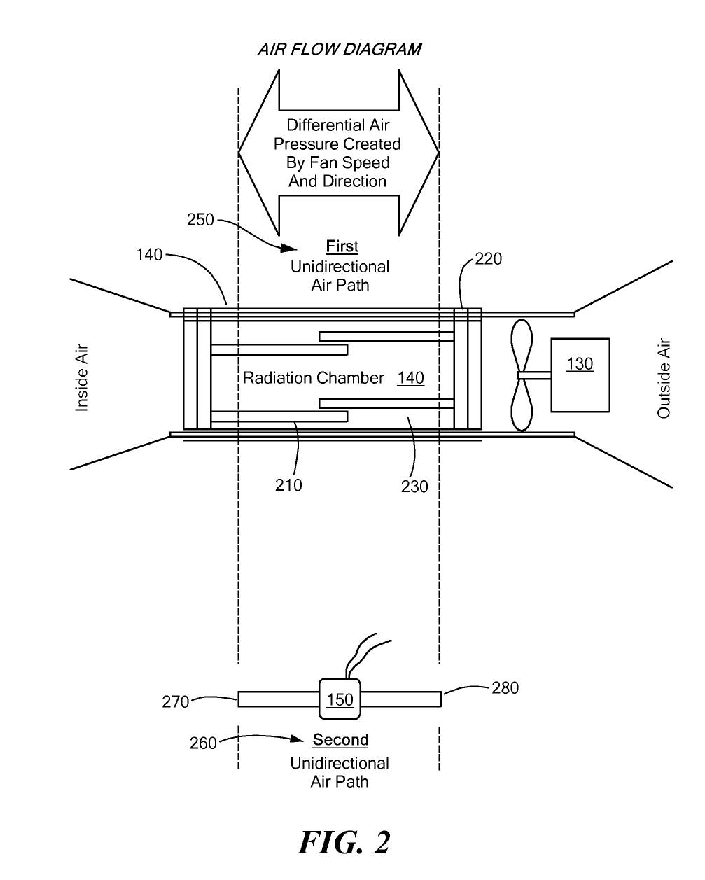

As show in FIG. 2, the germicidal radiation chamber 140 may contain ultraviolet lamps 210 that radiate at a wavelength of approximately 253.7 nanometers. UV radiation at 253.7 nanometers has been proven to inflict the greatest amount of damage on living and dormant micro-organisms. For example, at 253.7 nanometer wavelength, UV testing on influenza indicates a 90% kill ratio with severe damage (sufficient to neutralize) inflicted on the remaining 10%. The targets of the germicidal radiation chamber 140 include, but are not limited to: viruses, bacteria, fungus, mold, and spores. Although a 253.7 nanometer wavelength is used as an example, the UV wavelength can be adjusted to maximize the damage to any one species of micro-organisms.

The radiation chamber 140 may also provide access to the UV lamps 210 so that a user may replace the UV lamps 210 when needed. The user can install the UV lamps 210 from outside of the germicidal radiation chamber 140 so that they need not disassemble the chamber 140. The access to the UV lamps 210 may include a kill switch that shuts off the system 110 to prevent a user from accessing the UV lamps 210 during operation. Alternatively, the germicidal radiation chamber 140 may be a cartridge design that a user can completely remove and replace at a remote location. In some embodiments, the germicidal radiation chamber 140 may include multiple UV lamps with varying wavelengths to target different types of airborne particulates or micro-organisms.

As mentioned above, the germicidal radiation chamber 140 can be removable. In embodiments containing a removable radiation chamber 140, the system may also include an interlock switch that is electrically connected to the radiation chamber 140. The interlock switch can verify that the radiation chamber 140 is installed correctly and, in the event of incorrect installation, cut off the main power to the system 110, for example, to prevent accidental exposure to UV light.

Destruction and neutralization of micro-organisms using UV light depends on the amount of UV light that the micro-organisms are exposed to and the exposure time. To increase the amount of exposure, the inside surface of the germicidal radiation chamber 140 may contain a reflective coating 230. The reflective coating 230 reflects the UV light within the chamber, exposing the micro-organisms to greater amounts of UV light and, thus, increasing the micro-organism kill and neutralization ratios. Additionally, in some embodiments, the exposure time may be increased by slowing down the air flow within the germicidal radiation chamber 140. A laminar air flow through chamber 140 can assure that the resident time and exposure is uniform and equal throughout chamber 140. To further increase the exposure and residence time, the chamber 140 should be as large as possible within the constraints of overall size of the system 110. Dead spots in the airflow should be minimized.

UV light is hazardous and should be contained within the germicidal radiation chamber 140 and system 110. To prevent UV light from escaping and to help prevent accidental exposure to UV light, the germicidal radiation chamber 140 may include baffles 220 at one or both ends. Additionally or alternatively, the airflow path of the system 110 may be blackened to prevent UV reflection through the system inlet or outlet.

A differential-air-pressure transducer 150 can measure the air pressure at the inlet and outlet of the system 110. The differential-air-pressure transducer 150 may sample and measure the air pressure of the inside air through a closed space air port 270 and can measure the outside air pressure through an outside air port 280. The system 110 may contain pressure-tight connections between the differential pressure transducer 150 and air ports 270, 280. The outside air port 280 may contain provisions to prevent blockage from freezing weather and other variables such as insects. If the system 110 is not installed in a window, the outside air port 280 may also include an extension to reach the outside environment. In some embodiments, the differential-air-pressure sensor 150 can be a hot-wire or solid state anemometer. In other embodiments, a pressure transducer 150 may be located in a second airflow path 260. As shown in FIG. 2, the second airflow path 260 may be separate and distinct from the first airflow path 250, which contains the germicidal radiation chamber 140.

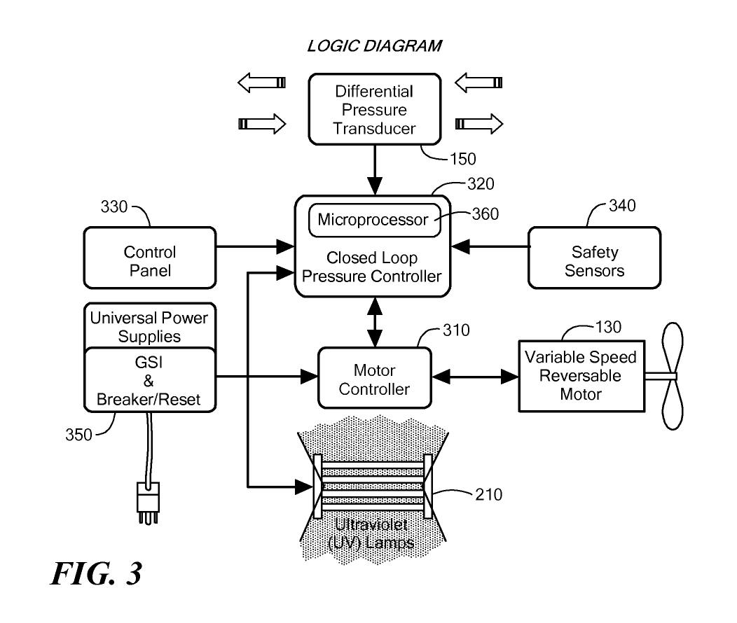

As shown in FIG. 3, the system 110 may include a closed-loop controller 320. that is connected to the differential-air-pressure transducer 150, and a motor controller 310. The closed-loop controller 320 may monitor the pressure differential between the system inlet and the system outlet and, based on the pressure differential, adjust the speed of the fan 130 via the motor controller 310. By controlling the speed of the fan 130 via the motor controller 310, the closed-loop controller 320 is able to control the pressure within the closed space 180. The motor controller 310 may work on all voltages and cycles, and have a selectable voltage switch. In embodiments containing multiple fans, the motor controller 310 may have a different controller power situation for each unit.

During startup, the closed-loop controller 320 may be configured to expect a worst case scenario and bring the fan 130 to full speed. In response to a power interruption to the system 110, the closed-loop controller 320 may provide an orderly shut down and start up process.

The closed-loop controller 320 may include a microprocessor 360. The microprocessor 360 may compare the differential-air-pressure transducer 150 output to a setpoint inputted by the user via a control panel 330 (discussed below) or pre-programmed into the system 110. The microprocessor 360 may then adjust the speed of the fan 130 to maintain the pressure within the closed space 180 at the setpoint value. When the system 110 is operating out of set point conditions, the closed-loop controller 320 may trigger an alarm to alert the user/home owner.

The closed-loop controller 320 may also include a second control band capable of recognizing when a door 170 (FIG. 1) is opened or when another change in condition within the space 180 occurs. The closed-loop controller 320 may then respond to such a condition by taking the fan 130 to full speed and then closing on a setpoint. The closed-loop controller 320 may also set a dead band to prevent the fan 130 from hunting.

In other embodiments, the closed-loop controller 320 may verify the presence of UV light and control the intensity of the UV radiation based on the air flow through the system 110. For example, the closed-loop controller 320 may control the intensity of the UV radiation by turning on all UV lamps 210 for maximum radiation, or by turning on one UV lamp at a time to perform a step function of radiation levels. The closed-loop controller 320 may also recognize if a UV lamp fails and switch the power to a functioning lamp.

In some embodiments of the present invention, the closed-loop controller 320 may contain a software port (not shown). The software port allows a user to download new software revisions (including new/updated setpoint values) and to test individual functions of the system 110.

In further embodiments, the system 110 may contain a control panel 330 that, among other things, allows a user to input setpoints values into the control panel 330. The control panel 330 may also contain a switch (not shown) to allow the user to choose between either positive or negative room pressure. The switch can be either a mechanical switch, a key pad, or a key pad multiple digital code. In embodiments containing multiple fans, the control panel 330 may allow the user to select one of the fans to move in a different direction. Other functions of the control panel 330 include, but are not limited to, diagnosing one or all functions of the control system, and displaying when routine services, such as UV lamp 210 replacements, are needed. The control panel 330 may be available in multiple languages.

In accordance with other embodiments of the present invention, the system 110 may also contain safety sensors 340. The safety sensors 340 may include an audible or visible alarm. The safety sensor 340 and the associated alarm may be in communication with the microprocessor 360 and the closed-loop controller 320. After receiving a signal from the closed-loop controller 320, the safety sensor 340 may trigger the alarm if the system 110 is not operating at the setpoint value or when system components are not functioning properly.

A universal power supply 350 supplies power to the system 110. The power supply 350 contains a GSI and a breaker reset and may be plugged into a standard wall socket.

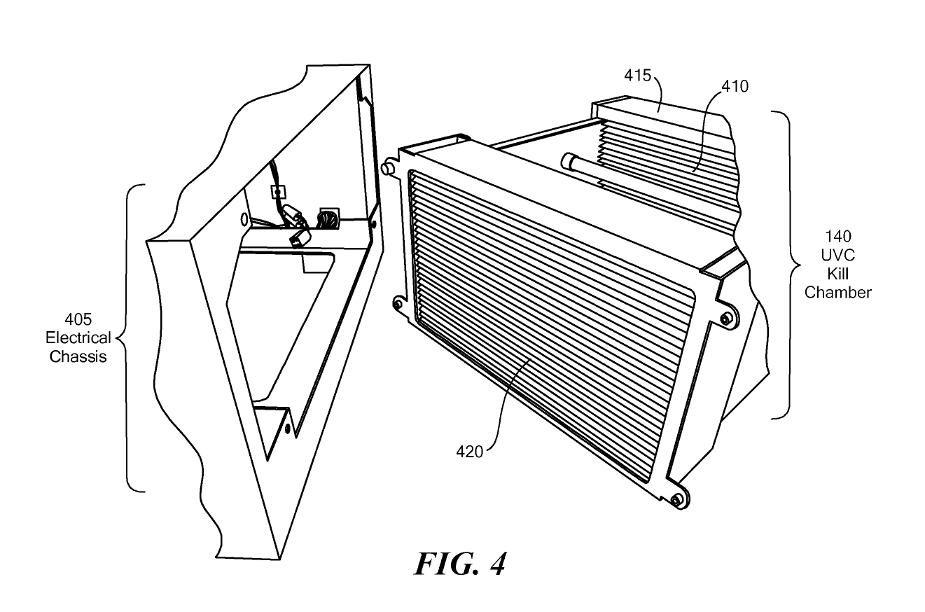

As shown in FIG. 4, the germicidal radiation chamber 140 can be contained within an electrical chassis 405. In such embodiments, a user can essentially slide the germicidal radiation chamber 140 into the electric chassis 405 to create the complete system 110. As discussed in greater detail below, the electrical chassis 405 houses many of the electrical and mechanical components of the system 110.

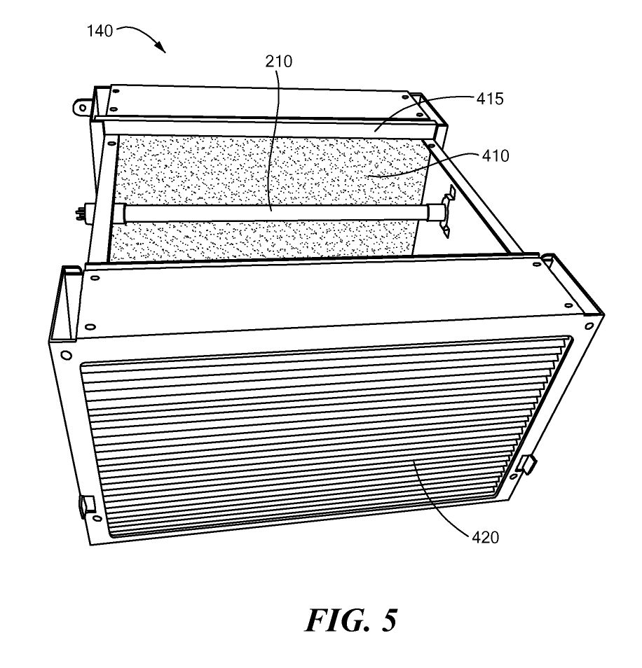

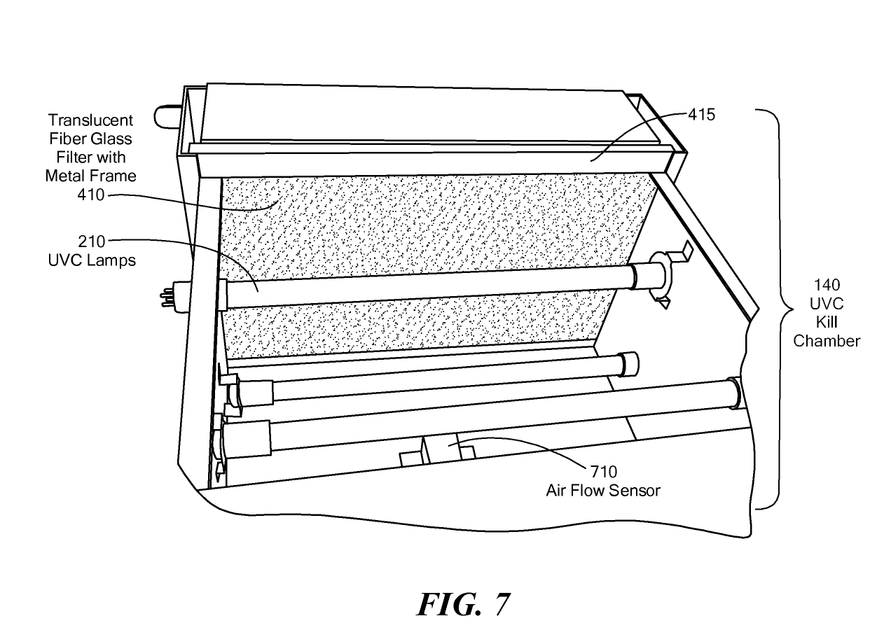

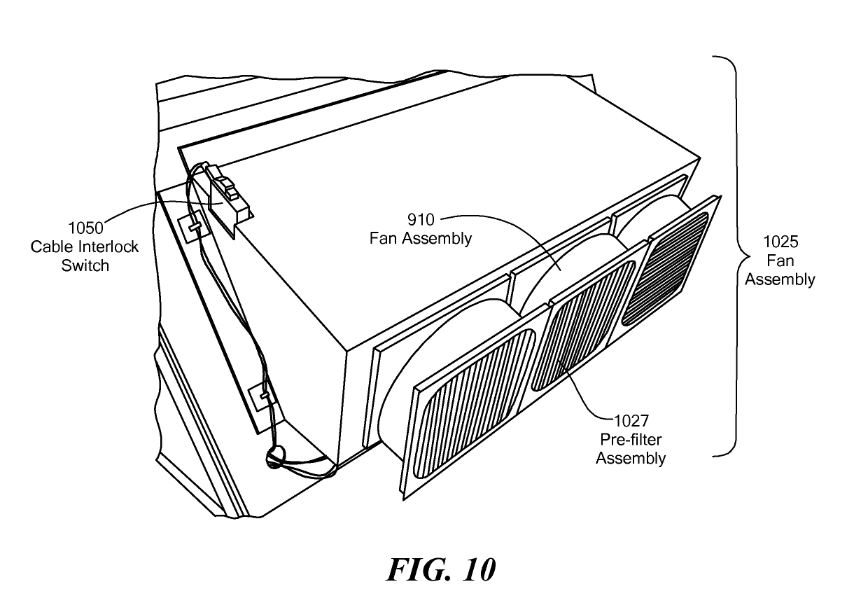

The system 110 may be a filter-less system or may include a HEPA filter 410. In filter-less embodiments, the UV light kills or neutralizes the micro-organisms as they pass through the germicidal radiation chamber 140. As shown in FIGS. 4 and 5, in systems 110 having filters, the HEPA filter 410 may be located at one or both ends of the germicidal radiation chamber 140. For example, the filter 410 may be at the opposite end of the germicidal radiation chamber 140 from the fans 910 (see FIGS. 7 and 10). To ease filter installation and replacement, the germicidal radiation chamber 140 may include slots that allow access to the filter 410. The addition of the filter 410 and two more sensors (an air flow sensor in the UVC chamber and a UVC level sensor in the UVC chamber, discussed in greater detail below) essentially make the system 110 a portable air cleaner and air sterilizer as well as a room isolation controller and a room containment controller.

In preferred embodiments, the filter 410 should be a translucent fiber glass HEPA filter. The translucent filter allows the UV radiation to pass through the filter, allowing the UVC radiation to kill the viruses as they move through the germicidal radiation chamber 140 and pass through the filter 410. In some embodiments, the filter may be pleated to increase the effective surface area of the filter. The pleated filters can be oriented such that the pleats are vertical, and the axis of the UV lamp 210 is transverse to the filter pleat axis. In preferred embodiments, the UV lamps 210 are co-planar.

The HEPA filter 410 will trap larger contamination, exposing the larger contamination to continuous irradiation by the high intensity UVC lamps 210. By doing so, the filter 410 allows for destruction of the larger particulates (which require greater amounts of irradiation to be killed), while maintaining a manageable system size and the flowrates needed for room isolation and containment. It is important to note that the UVC radiation will dissociate most organic particulates from the HEPA filter 410, creating a self-cleaning filter.

The filter 410 and filter frame 415 (FIG. 7) should be constructed from materials that are resistant to UVC radiation. For example, the filter 410 may be translucent fiber glass, and the filter frame 415 may be metal.

The entrance to the germicidal radiation chamber 140 can also include a UVC light baffle and a flow straightener 420. As discussed above, the UVC light baffles prevent UV light from exiting the germicidal radiation chamber 140. As the name suggests, the flow straightener(s) 420 straighten the air flow through the system and may be used to reduce turbulence within the germicidal radiation chamber 140.

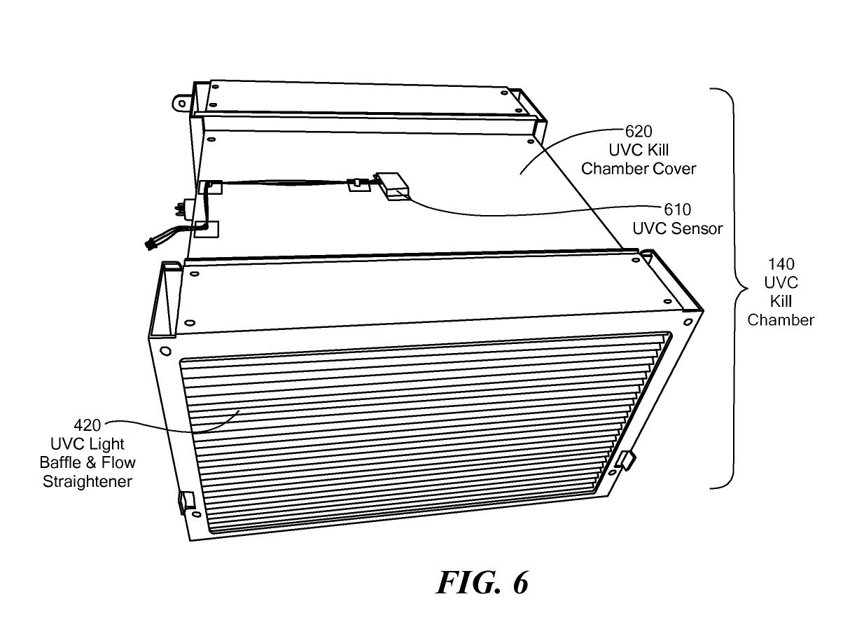

As shown in FIG. 6, the germicidal radiation chamber 140 can have a cover 620 that encases the germicidal radiation chamber 140. In addition, some embodiments of the present invention may also have a UV level sensor 610 located within the germicidal radiation chamber 140. The UV level sensor 610 can either be in or at the edge of the air flow. The UV level sensor 610 can transmit a signal to the microprocessor, which may control the fan speed or indicator lights based on the UV level sensor signal.

As shown in FIG. 7, the system 110 may also include an air flow sensor 710 located within the germicidal radiation chamber 140 (e.g., mounted to the inside wall of the chamber) and connected to the microprocessor. In preferred embodiments, the air flow sensor should 710 be a solid state sensor and co-linear with the air flow. In addition, the air sensor 710 should be shielded from the UV radiation to prevent damage to the air flow sensor 710. The air flow sensor 710 can send a signal to the microprocessor indicative of the air flow through the system. The microprocessor may then use this signal to modify the fan speed or control an indicator light (e.g., an alarm). In some embodiments, the air flow sensors 710 can be temperature compensated.

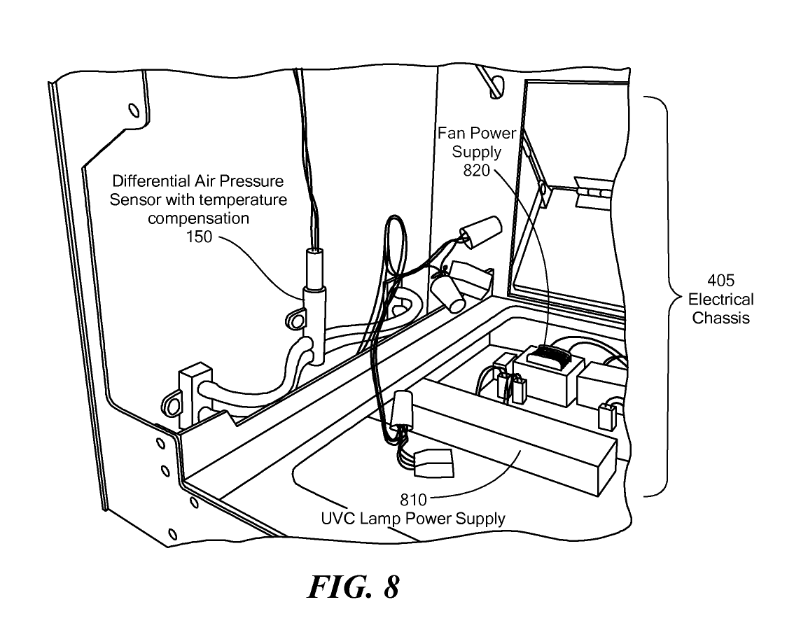

In addition to the above described components, the electrical chassis 405 can also house the UVC power supply 810 and the fan power supply 820 (FIG. 8). The electrical chassis 405 can also house the differential air pressure sensor 150. In a similar manner to the flow sensors 710, the differential air pressure sensor 150 can be temperature compensated.

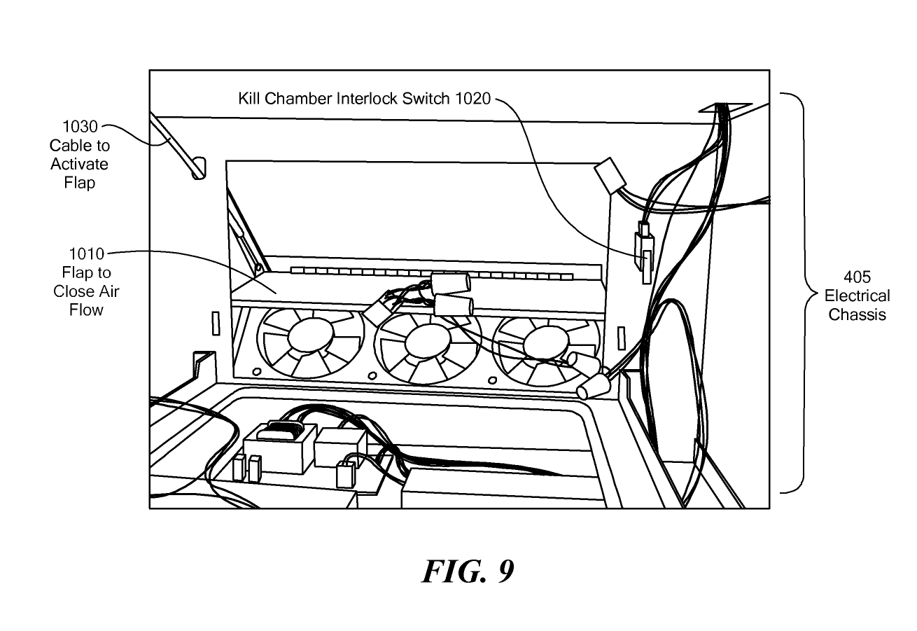

As shown in FIG. 9, to improve system storage and prevent debris, dirt, and other objects from collecting within the system 110, the system 110 may also have a cover 1010 that closes off the air flow when the system 110 is not in use. The cover 1010 may be, for example, a slide or a flap made from an insulating material. In some embodiments, the system may include a cover interlock switch 1020 electrically connected to the cover 1010 to sense the position of the cover 1010 (e.g., whether the cover is open or closed). The cover interlock switch 1020 may also be electrically connected to the microprocessor such that it prevents system operation when the cover 1010 is closed. For example, the interlock switch 1020 may send a signal to the microprocessor 360 indicating that the cover 1010 is closed.

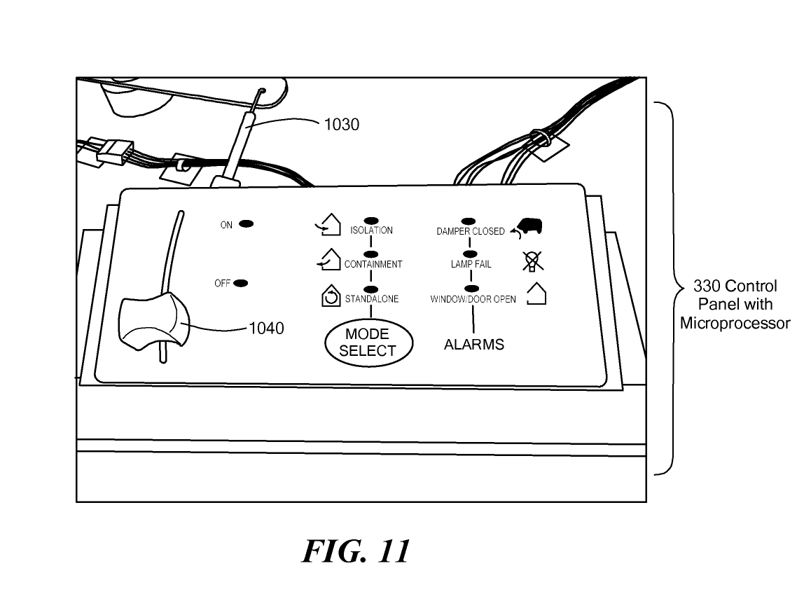

In some embodiments, a cable 1030 can be used to activate (e.g., open and close) the cover 1010. The position of the cable 1030 can act as the on-off switch for the system. For example, when the cable position corresponds to an open cover, the system 110 is on. Conversely, when the cable position corresponds to a closed cover, the system 110 is off. Like the cover 1010 itself, the cable 1030 can also be electrically connected to a cable interlock switch 1050 (FIG. 10) to sense the position of the cable 1030. A user can adjust the position of the cable 1030 (e.g., open and close) using a knob 1040 located on the system control panel 330 (FIG. 11).

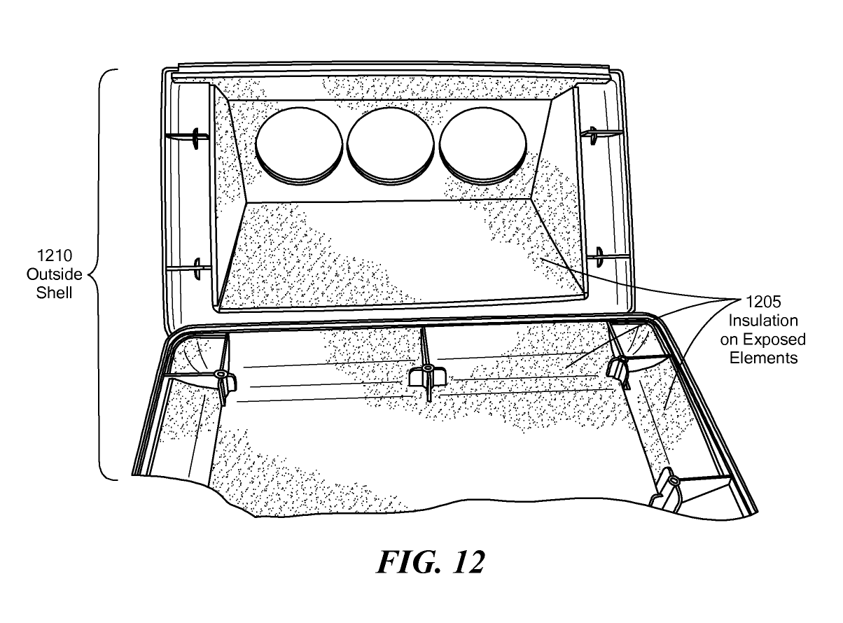

As shown in FIG. 10, the system can have a fan assembly 1025 attached to the electrical chassis 405. The fan assembly can have any number of fans (FIG. 10 shows 3 fans) that create the air flow through the system. As mentioned above, the fan speed can be controlled based on a number of criteria including, but not limited to, pressure differential, set points, and amount of UV light. The fan assembly 1025 can have a pre-filter assembly 1027 that covers each of the fans. The pre-filter assembly 1027 prevents larger objects, debris, or small animals from entering the system 110. In some embodiments, the portion of the system 110 exposed to the outside elements may have insulation 1205 (FIG. 12).

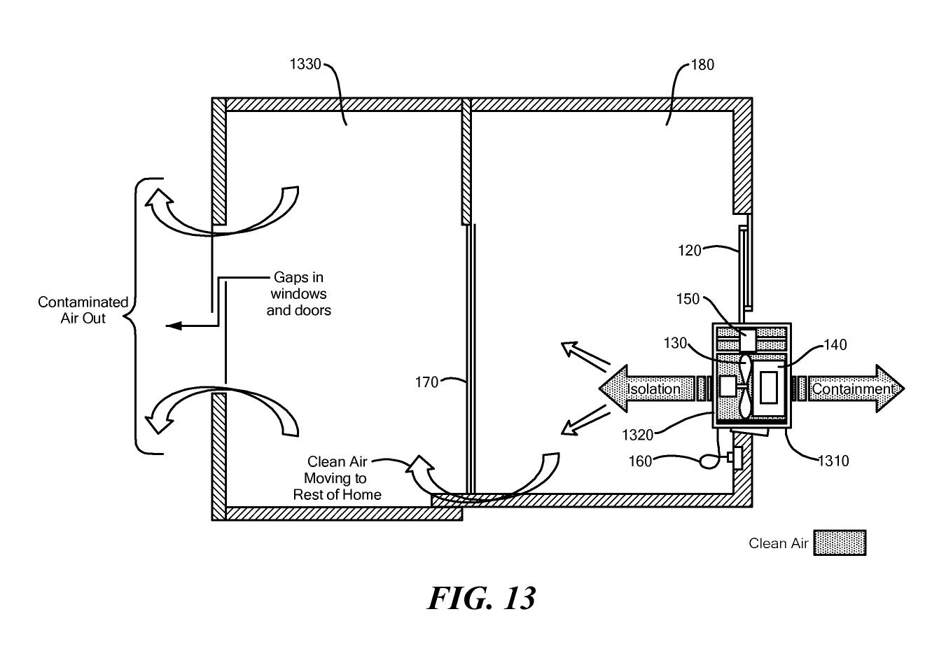

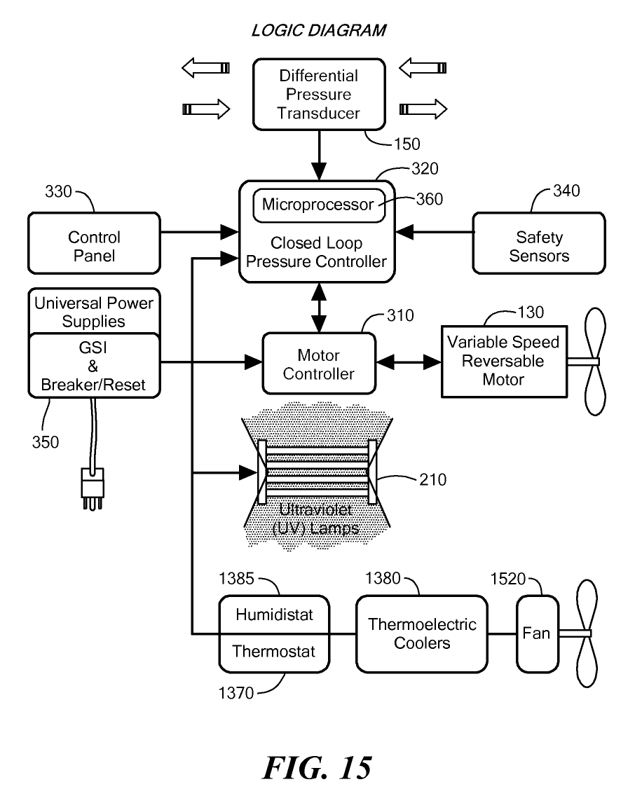

FIG. 13 shows an alternative embodiment of an air-pressure-isolation system 1310 in accordance with additional embodiments of the present invention. Like the system 110 shown in FIG. 1, the system 1310 shown within FIG. 13 may also be a through window, “plug and play” type system. As such, the system 1310 can similarly transform a closed space 180 into either an isolation or a containment room by placing the system 110 into a window 120 and plugging a power cord 160 into a standard wall socket. As shown in FIGS. 15-17 (discussed in greater detail below), the inward facing side 1320 of the system 1310 may have a stylish design so that it does not negatively impact the aesthetics of the closed space 180. The outward facing side of the system 1310 may have a design that is suitable for exposure to the environment.

In the isolation and/or containment configuration, the operation of the system 1310 shown in FIG. 13 is similar to that of the system 110 shown in FIG. 1. For example, in an isolation configuration, the variable speed fan(s) 130 force(s) clean air into the closed space 180, resulting in a positive pressure within the closed space 180. In order to produce a constant positive pressure consistent with surgical sites and clean rooms, the system 110 may control the air flow into the room, by varying the speed of the fan, to match the air flow out of the room through gaps around windows and doors. In the containment configuration, the variable-speed fan 130 forces air out of closed space 180, resulting in a negative room air pressure. In either orientation, a germicidal radiation chamber 140, located within a closed airflow path, cleans the air as it passes through system 110.

As discussed in greater detail below, the closed space 180 may be a room of a home, and the door 170 of the closed space 180 may lead to another room 1330 and/or the reminder of the home/building. In such instances, as also discussed in greater detail below, the system 1310 may be used to improve the air quality within the household.

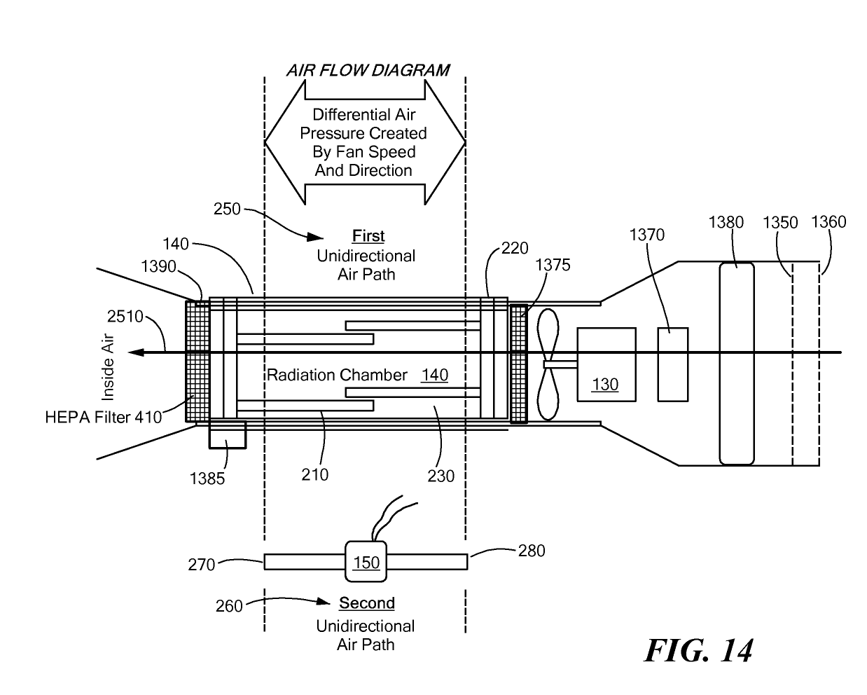

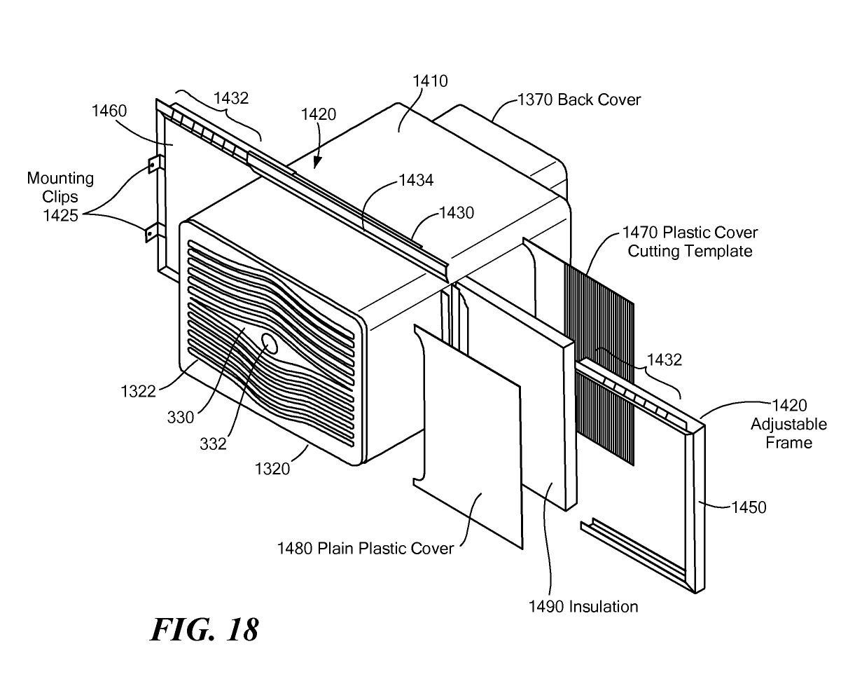

As show in FIG. 14, in addition to the germicidal radiation chamber 140 containing the ultraviolet lamps 210, the system 1310 may also include an intake plenum 1340 upstream of the fan 130. The intake plenum 1340 may include a pre-filter, a carbon filter 1350, and a bug screen 1360 to prevent debris, dirt, bugs, and other objects from collecting within/entering the system 1310. A back cover 1370 (FIG. 18) may be used to cover the components at the intake of the system 1310 (e.g., the back cover 1370 may be used to cover the intake plenum 1340). In the absence of the intake plenum 1340, the fan assembly 1025 can have a pre-filter assembly that covers the fan(s) (e.g., similar to that described above). The pre-filter assembly prevents larger objects, debris, or small animals from entering the system 1310.

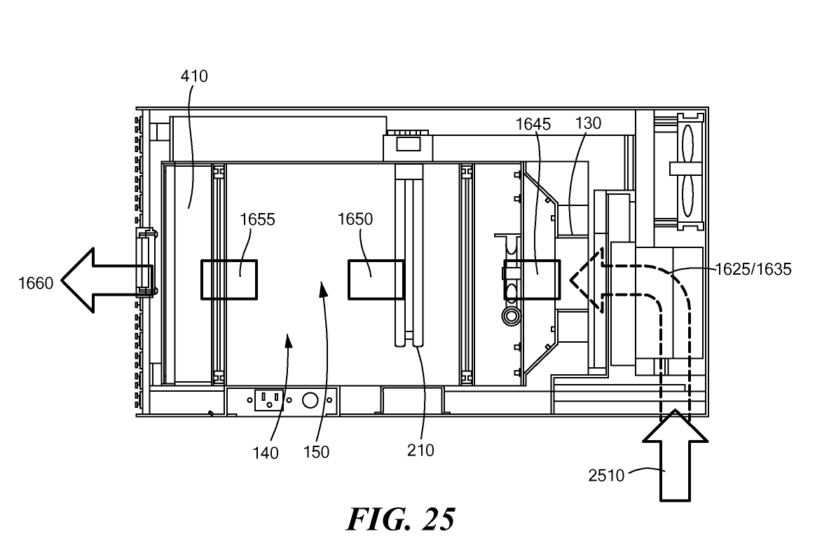

Additionally, in some embodiments, the system 1310 may have a thermostat 1370 located within the path of the external air 2510 (FIG. 25) drawn into system 1310. The thermostat 1370 may be connected to a heater/cooler 1380 (e.g., a reversible thermoelectric device). The thermoelectric device can pre-heat and/or pre-cool the air entering the system 1310 (e.g., the drawn air) based on a signal from the thermostat. For example, the thermoelectric device 1380 can heat and/or cool the drawn air based upon the temperature of the incoming air measured by the thermostat 1370 and/or the room temperature within space 180.

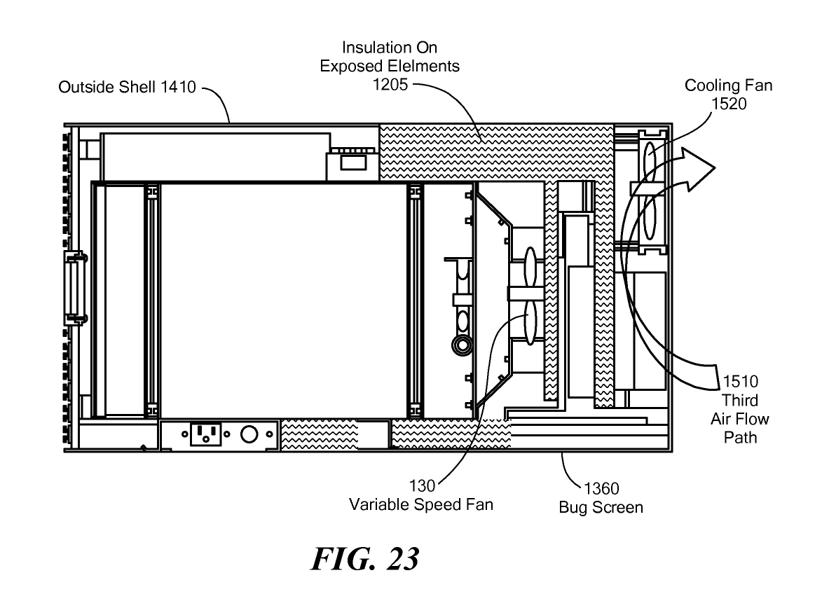

As shown in FIGS. 14 and 15, in embodiments containing the thermoelectric device 1380, the thermoelectric device 1380 may have one side exposed to the air flow 2510 and the other side exposed to a third air flow path 1510 (FIG. 23). The system 1310 may also include a second fan 1520 (e.g., a cooling fan) in the third air path 1510 (e.g., to draw air through the third air path 1510). The second fan 1520 may be activated when power is supplied to the thermoelectric device 1380.

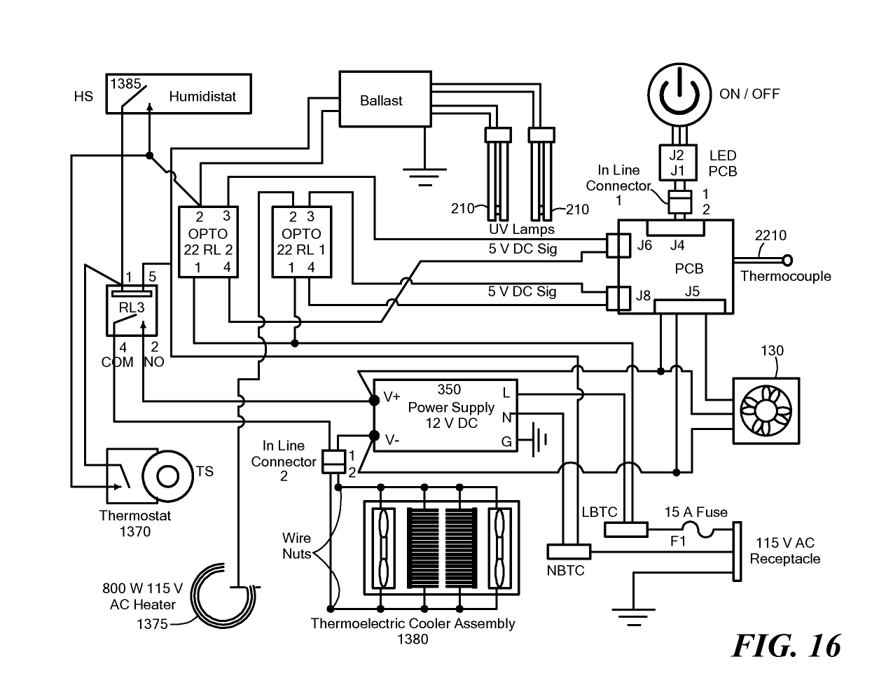

In order to sense/measure the humidity within the air flow path, some embodiments may also include a humidistat 1385 located in the air flow path (e.g., at the exit of the system 110). Like the thermostat 1370, the humidistat 1385 can also be connected to the thermoelectric device 1380 (FIGS. 15 and 16) to enable the thermoelectric device 1380 to cool (or pre-heat) and dehumidify the incoming air 2510. For example, based upon the temperature and humidity measurements, the thermostat 1370 and the humidistat 1385 can control the thermoelectric device 1380 to, in turn, control the temperature and humidity within the system 1310 and the temperature and humidity of the air exiting the system 1310 (e.g., into the space 180).

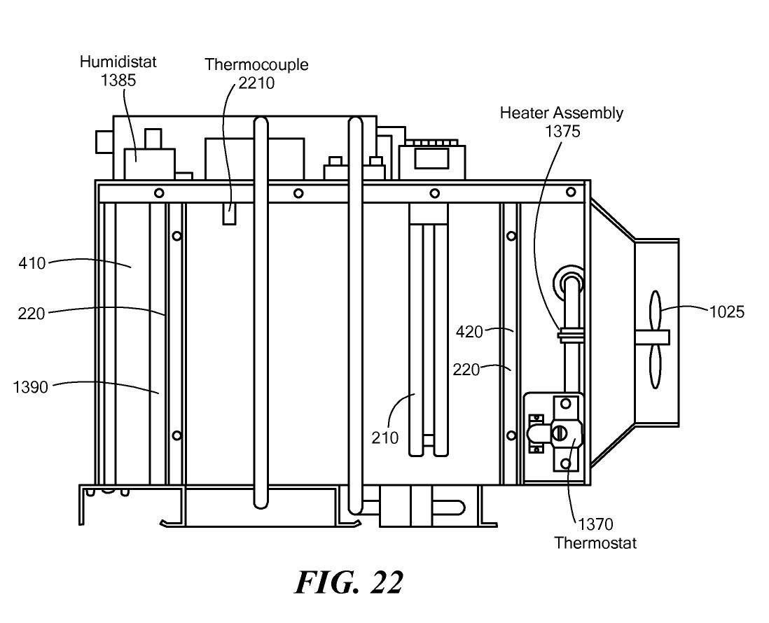

In addition to the humidistat 1385, thermostat 1370, and the thermoelectric device 1380, some embodiments may also have a main heater 1375 located just upstream of the radiation chamber 140, and a thermocouple 2210 (FIG. 22) located within the germicidal radiation chamber 140. The thermocouple 2210 may be connected to the closed-loop controller 320 which, in turn, can provide a modulated power to the main heater 1375. In this manner, in addition to dehumidifying and/or cooling the air passing through the system 1310, the system 1310 can also heat the air to prevent cold air (e.g., from outside of the home) from being introduced into the space 180/home 182.

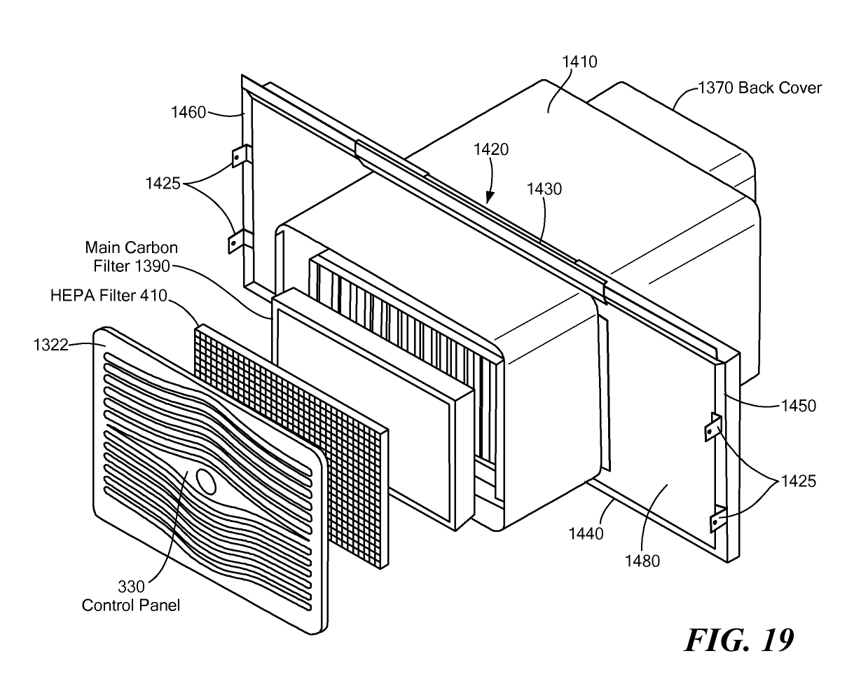

As discussed above, some embodiments of the present invention can include a HEPA filter 410 located at one or both ends of the germicidal radiation chamber 140. For example, as shown in FIGS. 14, 17, and 19, the HEPA filter 410 may be located at the outlet of the germicidal radiation chamber 140 (e.g., just behind the front cover 1322 and control panel 330). Additionally, upstream of the HEPA filter 410, some embodiments of the present invention can also include an additional carbon filter (e.g., main carbon filter 1390). As discussed in greater detail below, the filters (e.g., the HEPA filter 410, main carbon filter 1390, and pre-filter and carbon filter 1350) may be used to further clean and sterilize the air flowing through the system 1310.

Although the embodiments described above have control panels with a number of features (e.g., a switches, key pads, etc.), other embodiments can have a simpler control panel 330. For example, in true “plug and play” systems 1310, the control panel 330 can merely include an on/off button 332. As the name suggests, the on/off button 332 can be depressed by the user to turn the system 1310 off and on. All other control and operating conditions of the system 1310 (e.g., temperature, humidity, etc.) can be pre-programmed and automatically controlled by the system 1310. Additionally or alternatively, the control panel 330 can also include temperature and humidity controls (not shown) that allow the user to set a desired temperature and/or humidity of the air exiting the system 1310.

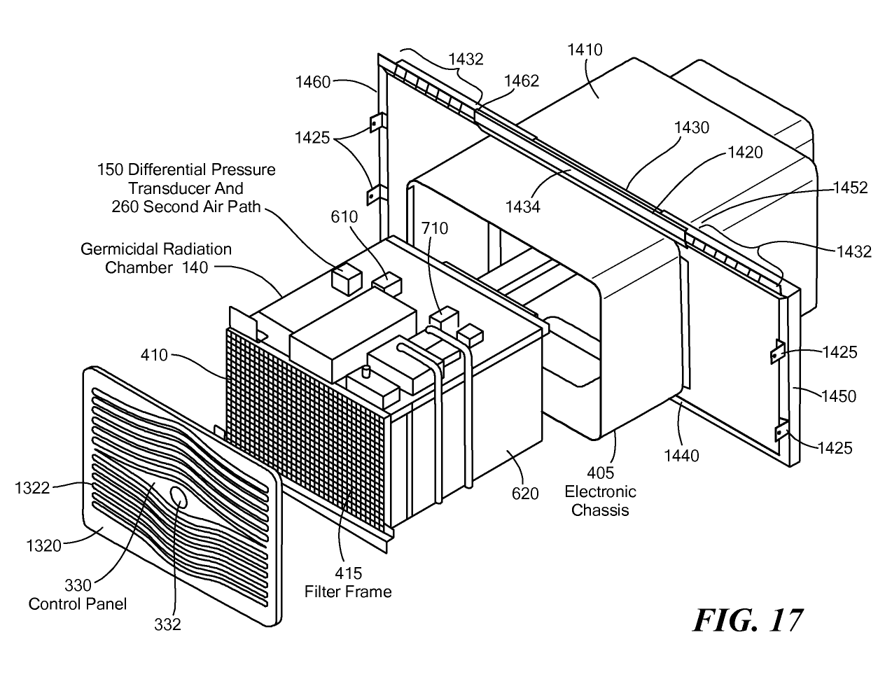

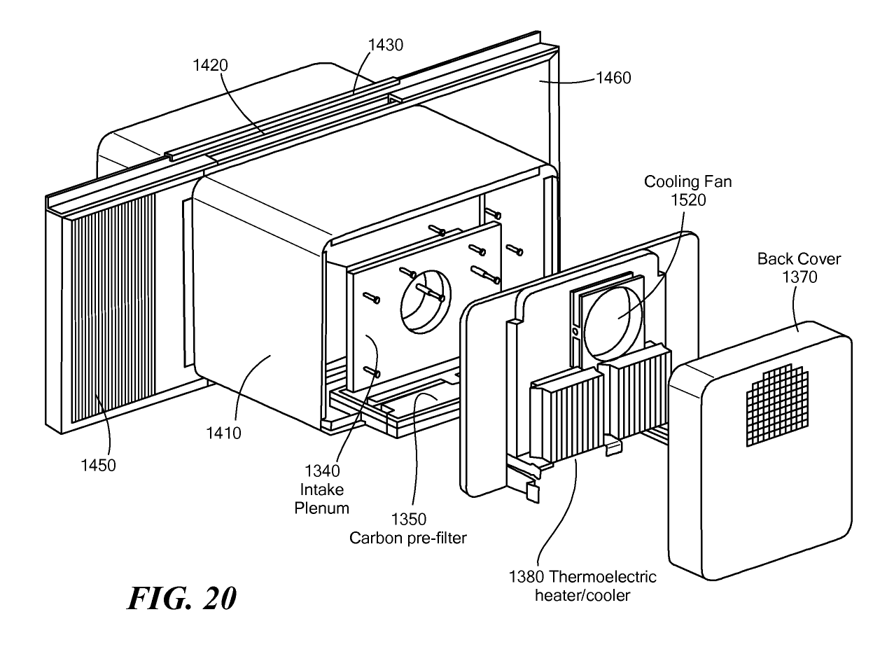

As shown in FIGS. 17-20, the system 1310 (or the system 110) can include an expandable frame 1420 extending around the periphery of the outside shell 1410 (or the electronic chassis 405). As mentioned above, various embodiments of the present invention can be configured for through window installation. To that end, the expandable frame 1420 can provide for a better fit in through-window installations. For example, the expandable frame 1420 can expand to the size of the window (e.g., the window frame) in which the system 1310 is installed. The expandable frame may include a soft gasket for sealing against the window sill, window frame, and the system shell.

As best shown in FIGS. 17-19, the frame 1420 can include a top rail 1430 extending across the top surface of the outside shell 1410, a bottom rail 1440 extending across the bottom surface of the shell 1410, and two adjustable side rails 1450/1460 that extend from the right and left sides of the shell 1410. The top rail 1430 of the frame 1420 can include a tape measure 1432 calibrated at half scale (½ inch to an inch) with the zero point 1434 at the center of the top rail 1430. The numbers may increase in both directions (e.g., to the left of the zero point 1434 and to the right of the zero point 1434).

Prior to installing the system 1310 into the window, the top rail 1430 and the two adjustable side rails 1450/1460 can be placed in an open window and expanded to fit the window frame. This, in turn, reveals the numbers on the measuring tape 1432. The top rail 1430 can then be moved so that the zero point 1434 is at the center of the window and the numbers at the ends 1452/1462 of the adjustable rails 1450/1460 match (e.g., so that the side rails 1450/1460 are equidistant from the shell 1410). The number showing at the ends 1452/1462 of the adjustable rails 1450/1460 can then be used to cut a plastic cover template 1470 (FIG. 18). The template 1470 can then be used to mark and cut plastic covers/panels 1480 and insulation 1490.

After cutting the cover/panels 1480 and insulation 1490, the individual installing the system 1310 can assemble the frame 1420 with the cut covers/panels 1480 and insulation 1490 located in the space between the side rails 1450/1460 and the sides of the shell 1410. The individual may then fasten the system 1310 to the window 120 using the mounting clips 1425. As mentioned above and as shown in FIG. 23, the portion of the system 1310 exposed to the outside elements may have insulation 1205.

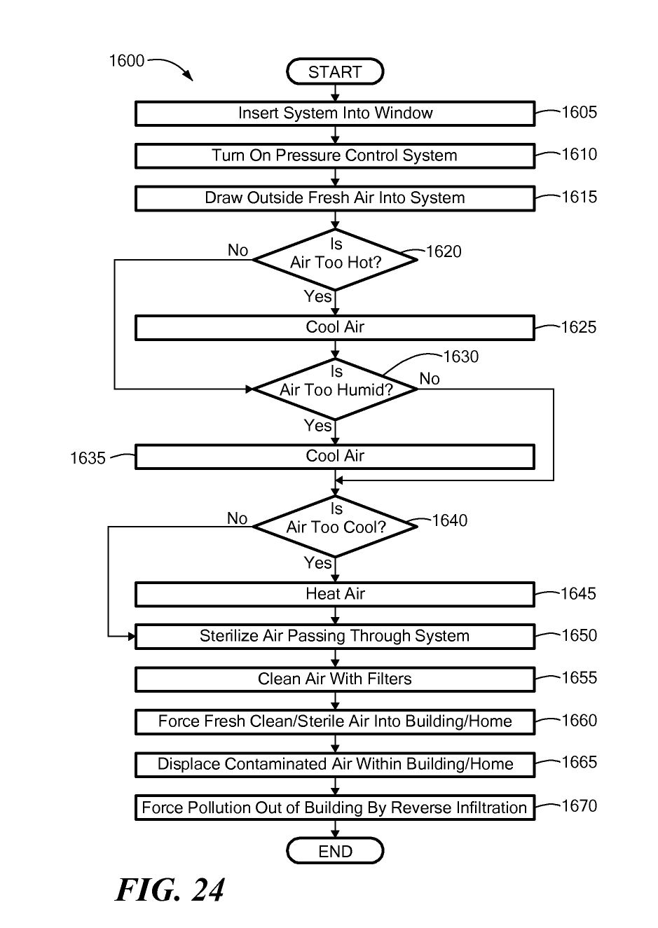

In addition to being used for creating the isolation and/or containment rooms discussed above, some embodiments of the pressure control system 1310 (and/or system 110) can also be used to improve the air quality within a household. For example, the control system 1310 may be placed within the window 120 of a room in a house 182, and can be used to replace contaminated air within the home with clean/sterile air. FIG. 24 shows one embodiment of a method of air replacement in accordance with the present invention. The location of the drawn air within the system 1310 during the various stages of the method discussed below is shown in FIG. 25.

According to the method 1600, the homeowner (or other individual), can install the air-pressure control system 1310 into a window of the home (Step 1605), and turn on the system (Step 1610). It is important to note that, instead of a window, the air-pressure control system 1310 may be installed into a doorway, or other opening within the home that allows the system 1310 to draw in air from outside of the home (e.g., any opening that passes through an exterior wall of the home).

Once installed into the window and turned on, the system 1310 will draw in air from the exterior of the home (Step 1615). In some embodiments, the air drawn from the exterior of the home can be conditioned to room temperature and humidity (e.g., the temperature and humidity within the home). For example, if the system 1310 determines that the temperature is above room temperature (e.g., using the thermocouple 2210) (Step 1620) or that the incoming air is too humid (e.g., using the humidistat 1385) (Step 1630), the system 1310 can cool the air (Steps 1625 and 1635) using the coolers within the thermoelectric device 1380. Conversely, if the system 1310 (e.g., the thermocouple 1385 and/or thermostat 1370) determines that the incoming air is too cold (Step 1640), the system 1310 activate the main heater 1375 to heat the drawn-in air to room temperature (Step 1645).

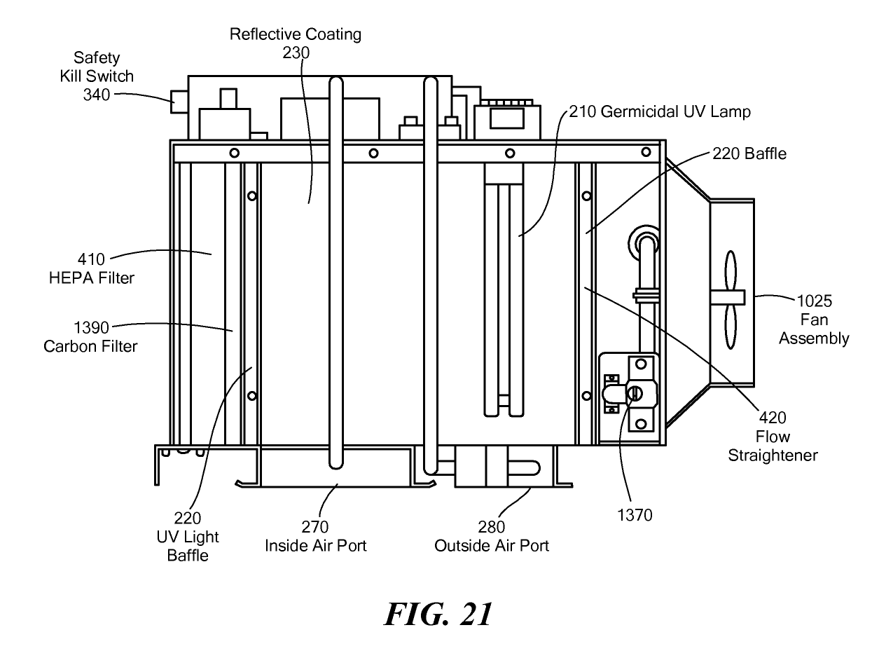

As mentioned above, some embodiments of the system 1310 can have a germicidal radiation chamber 140 and/or one or more filters (e.g., the HEPA filter 410, the main carbon filter 1390, and/or the pre-filter and carbon filter 1350) located on either side of the germicidal radiation chamber 140 (FIGS. 14, 17, 19 and 21). To that end, some embodiments of the control system 1310 can sterilize (Step 1650) (e.g., in those embodiments that include the germicidal radiation chamber 140), and clean (Step 1655) (e.g., in those embodiments that include the filter(s)) the air as it passes through the airflow path and the germicidal radiation chamber 140. For example, as the drawn air passes through the germicidal radiation chamber 140, the UV light can destroy bacteria and micro-organisms (e.g., viruses, bacteria, fungus, mild, and spores) within the air. Additionally, the filter(s) 410 can remove/trap micro contamination within the airflow.

The cleaned and/or sterilized air may then be introduced into the home (e.g., into the room/space 180 in which the air-pressure control system 1310 is located) (Step 1660). As the cleaned/sterilized air is introduced into the room, an equal volume of contaminated air within the room/home is displaced (e.g., for each cubic foot of air that is introduced into the room/home, a cubic foot of air is displaced out of the room/home) (Step 1665). The air replacement will begin at the entrance point of the cleaned/sterile air (e.g., at the outlet of the air-pressure control system 110) and will gradually move throughout the room into the adjoining room 1330 and the remainder of the home 182. As additional contaminated air is displaced and replaced by clean/sterile air, the airborne contamination throughout the entire living space is forced out of the building/home 182 (Step 1670) (e.g., by reverse infiltration), and the overall contamination level within the building/home 182 is reduced.

It is important to note that the germicidal radiation chamber 140 and the filter(s) can, together, remove substantially all of the contamination within the air drawn from outside of the home. For example, the output of the air-pressure control system 1310 (e.g., the air introduced into the room/home) can contain substantially no particles ranging in size from 5.0 microns to 0.3 microns. Therefore, as the contaminated air is displaced and replaced with the air being output by the air-pressure control system 1310, the air quality within the home improves, and any harmful particulates and/or volatile organic compounds (VOCs) (e.g., any airborne contamination) are removed from the home.

As mentioned above, the air-pressure control system 1310 can control the speed of the fan 130 about a set point using the microprocessor 360 and/or closed-loop controller 320. The set point can be preset at the factory during manufacturing or the set-point can be input into the system by the end user (e.g., before or just after inserting the control system 1310 into the window). For example, the set-point can be preset (or set by the end user) to control the fan to maintain an airflow rate of between 24 cubic feet per minute (CFM) and 75 cubic feet per minute (CFM). The airflow rate set points of between 24 CFM and 75 CFM are merely examples, and the airflow rate can be set to any suitable flow rate. In some embodiments, the flow rate can be dependent upon the size of the room/home, the level of contamination within the home, the time desired to clean the room and/or home, and/or the expected level of contamination of the air outside the home (e.g., the air being drawn into the system 1310).

### Exemplary Study:

Using an air-pressure control system in accordance with various embodiments of the present invention, a study was conducted to explore the ability of some embodiments of the present invention to reduce airborne contamination within the home by replacing contaminated air within the home with clean air (e.g., Air Replacement Technology (A.R.T.™)). The study compares A.R.T. to a recent national study that used air filtration products to reduce airborne contamination and quantify the health benefits. The pilot study was conducted in seven homes identified by the Massachusetts Support Group of the Alpha 1 Association.

The study and the Air Replacement Technology (A.R.T.) is based on two scientific principles—(1) that two bodies cannot occupy the same space at the same time, and (2) the effects of differential air pressure creating air flow. Based upon the above, it was determined that, for each cubic foot of clean, sterile air pushed into the home, a cubic foot of contaminated air is forced out. Each cubic foot of air leaving the home will contain contamination which will include mixtures of particulate and gases (triggers).

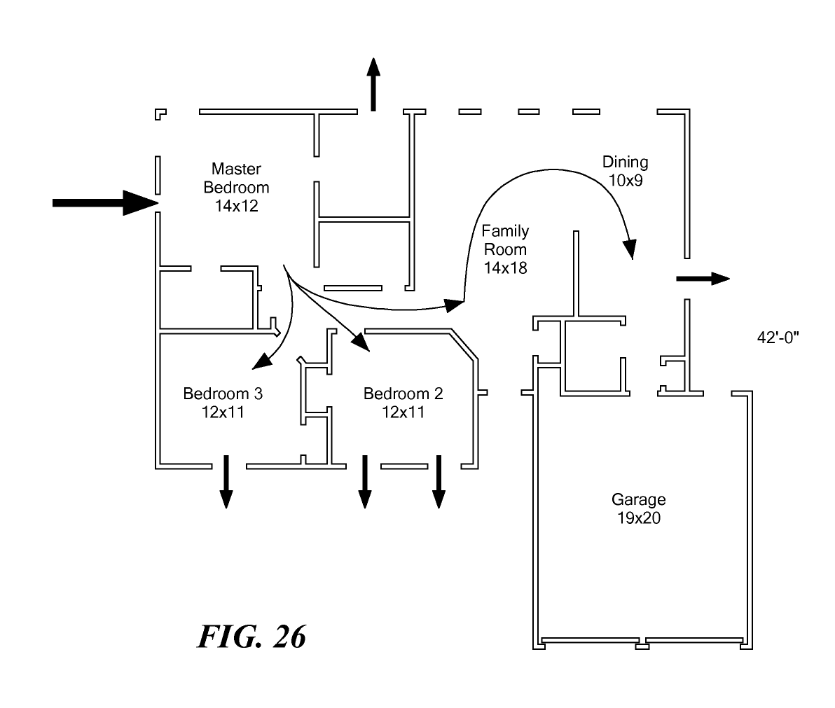

The process of air replacement technology begins at the entrance point of fresh sterile air (e.g., at the exit of the air-pressure control system 110/1310) and gradually moves throughout the home, eventually reducing airborne contamination throughout the entire living space (FIG. 26). It is important to note that, in contrast to air replacement technology, traditional re-circulating air filter products are designed to address particulate contamination and do not address the issue of introducing clean, fresh air or removing volatile organic compounds (VOC's).

The objective of the study was to determine the effectiveness of air replacement technology in a typical home setting. Air-pressure control systems 110/1310 were installed in the homes of seven members of the Massachusetts Support Group of the Alpha 1 Association. The homes varied in style, size and occupancy, with some including pets. The homes were constructed between 1960 and 2000 and heated with forced hot water or forced hot air. A requirement of the study was the availability of a double hung window to accommodate the system installation. The majority of the installations occurred in late summer to include both the fall allergy season and part of the winter heating season,

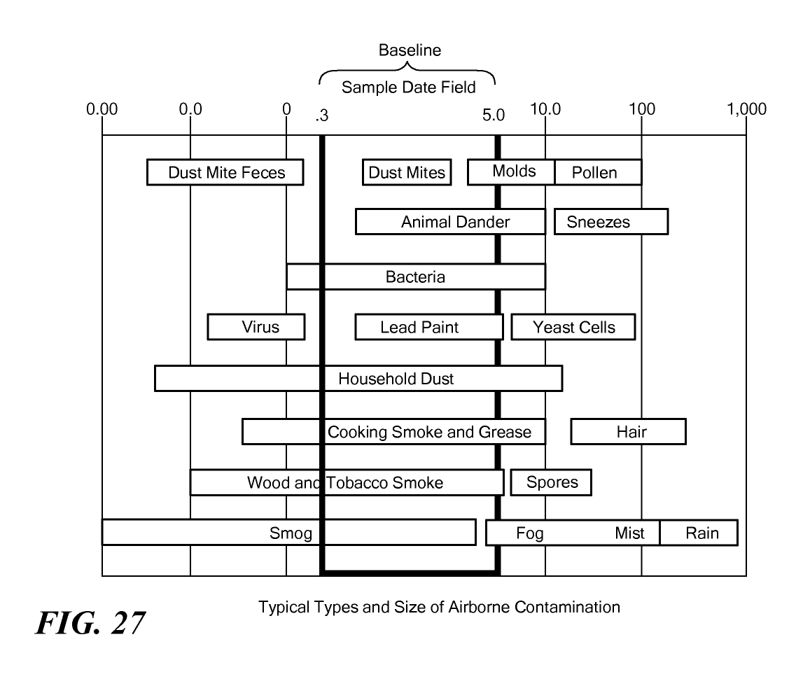

Once installed, the computer/controller of each of the installed systems controlled the differential pressure to create a stream of conditioned, clean, fresh, sterile air into the home, which, in turn, displaced all sizes and types of airborne micro-contamination, VOC's, and harmful gases. FIG. 27 shows a distribution of harmful pathogens. The airborne particulate levels were monitored in the size range typical of pathogens responsible for respiratory exacerbation. Baseline indoor air pollution data was collected at the time of installation of the air-pressure control system and measured monthly in each home for an average of four months.

All particle count data was taken with a MetOne GT-321 Hand Held Particle Counter. The data taken at installation and throughout the study includes five different particle sizes from 5.0μ to 0.3μ. All site visits verified that the clean, fresh sterile air entering the room from the air-pressure control system contained zero particles from 5.0μ to 0.3μ. Particle counts were also taken at the center of the room in which the air-pressure control system was installed, and in a kitchen or living room chosen by the participant.

The data presented below focuses on the most dangerous particle size (0.3μ), and all calculated averages are based on concentrations of 0.3μ particles. During installation data was taken in all homes at both the first and second location. The data was then averaged to determine a baseline concentration of 1,231,493 at 0.3μ particles per cubic foot at the first location, and 858,516 at 0.3μ particles per cubic foot at the second location. At each subsequent visit, the data from each location was averaged and compared to the baseline data for those locations and reported as a percent of particulate reduction. Table 1 shows the percent particle reduction at location one and Table 2 shows the percent particle reduction at location two.

TABLE 1
Percent Particle Reduction at Location #1

| | At Time of Install | Average | Average | Average | Average | Average Particle | Average |
|---|---|---|---|---|---|---|---|
| | Average of | Reading | Reading | Reading | Reading | Count for | Percent (%) |
| | 7 Homes | Visit #1 | Visit #2 | Visit #3 | Visit #4 | visits 2-7 | Particle Reduction |
| Particle | Zero | Zero | Zero | Zero | Zero | Zero | Zero |
| Count @ | | | | | | | |
| System | | | | | | | |
| Particle | 1,231,493 | 383,206 | 304,610 | 291,030 | 519,309 | 374,539 | 70% |
| Count in | | | | | | | |
| Room Cleaned | | | | | | | |
| by System | | | | | | | |

TABLE 2
Percent Particle Reduction at Location #2

| | At Time | Average | Average | Average | Average | Average Particle | Average |
|---|---|---|---|---|---|---|---|
| | of | Reading | Reading | Reading | Reading | Count for | Percent (%) |
| | Install | Visit #1 | Visit #2 | Visit #3 | Visit #4 | visits 2-7 | Particle Reduction |
| Particle | 858,516 | 477,687 | 487,683 | 469,231 | 239,297 | 418,474 | 51.3% |
| Count in | | | | | | | |
| Additional | | | | | | | |
| Room | | | | | | | |

It is important to note that the choice of a cubic foot of air as a sample size has respiratory significance. In particular, the average adult inhales about one cubic foot of air per minute. The concentrations of dangerous 0.3μ particles tracked in this study have respiratory significance because (1) they float and stay airborne for days, (2) the 0.3μ size particles can travel deep into the lungs, and (3) they can be absorbed by the body and trigger respiratory inflammation.

The above data shows that air replacement technology provided the greatest improvement in indoor air quality at the point of installation. Throughout the study, the particle count of the replacement air delivered by the air-pressure control system was zero for particles between 5.0 and 0.3 microns. The zero particle count readings at the air-pressure control system were consistent for all homes for the duration of the study.

The effectiveness of the fresh sterile air being introduced into the air-pressure control system installed location varied with the greatest individual reduction in airborne particulate of 87% and an average 4 month group reduction of 70%. (See Table 1)

As mentioned above, data was also taken in all homes at a second remote location. The contribution of the supply of fresh sterile air flowing through the first location to the second location also varied. The greatest individual reduction in airborne particulate was 77% with an average 4 month group reduction of 51%. (See Table 2)

Two short studies were also conducted to determine how quickly a room responds to air replacement technology. In both cases, the room responded at a contamination reduction rate of approximately 1% per minute.

### Conclusions

The use of available air cleaners to determine the health benefits of reducing airborne particulate in the home was previously reported in a 2011 nationwide study funded by National Institutes of Health (NIH). The NIH study found that a 20% reduction of airborne particulate results in an 18% reduction of unscheduled hospital visits. It is important to note that the NIH study preceded the introduction of air replacement technology (A.R.T.) and the air-pressure control systems described herein. The NIH was constrained by the use of available air cleaning technology study, and specifically expressed disappointment in removing only 20% of the airborne particulates, leaving all other forms of airborne pathogens behind.

In sharp contrast, various embodiments of the present invention removed 70% of the particulates. Furthermore, based on the physics of particle disbursement, all forms of indoor air pollution were present in each cubic foot of air that left the home.

The data taken at the second location supports the concept of differential air pressure transporting the benefits of the clean fresh air to other parts of the home. The transport of clean, fresh air to other parts of the home is a vast improvement over the localized air cleaning limitations of re-circulating air filter cleaners.

Although various exemplary embodiments of the invention have been disclosed, it should be apparent to those skilled in the art that various changes and modifications can be made which will achieve some of the advantages of the invention without departing from the true scope of the invention. These and other obvious modifications are intended to be covered by the appended claims.

What is claimed is:

1. A method for reducing airborne contamination within a home comprising:
installing an air-pressure control system within a window of the home, the air-pressure control system including:
a system inlet,
a system outlet,
a variable-speed fan configured to operate at a speed,
a motor controller in communication with the fan and configured to control the speed of the fan,
a solid state anemometer configured to monitor an air pressure differential between the system inlet and the system outlet,
a closed-loop controller in communication with the motor controller and the solid state anemometer, wherein the closed-loop controller is configured to vary the speed of the fan based on the pressure differential between the inlet and outlet of the system, and
a germicidal radiation chamber located within an airflow path in the air-pressure control system, the germicidal radiation chamber including at least one UV light source;
drawing air from outside the home through the system inlet and the airflow path, the germicidal radiation chamber sterilizing the air as it passes through the airflow path; and
introducing the sterilized air into the home through the system outlet, the sterilized air displacing an equal volume of contaminated air within the home.

2. A method according to claim 1, wherein the air-pressure control system includes at least one filter located within the airflow path, the filter cleaning the drawn air by removing particulates from the drawn air as it passes through the filter.

3. A method according to claim 2, wherein the filter is located at a first end of the germicidal radiation chamber.

4. A method according to claim 3, wherein the air-pressure control system further includes a second filter located at a second end of the germicidal radiation chamber.

5. A method according to claim 1, wherein introducing the sterilized air into the home includes introducing the sterilized air at a flowrate between 20 cubic feet per minute and 75 cubic feet per minute.

6. A method according to claim 1, wherein the air-pressure control system removes volatile organic compounds from the drawn air.

7. A method according to claim 1, wherein the introduced air contains substantially no particles between 5.0 microns and 0.3 microns.

8. A method according to claim 7, wherein the particles include at least one of dust mites, animal dander, bacteria, lead paint, household dust, cooking smoke and grease, wood and tobacco smoke, and smog.

9. A method according to claim 1, wherein displacing the contaminated air within the home improves the air quality within the home.

10. A method according to claim 1, wherein the contaminated air includes at least one of volatile organic compounds, airborne micro-contamination, harmful gases, dust mites, animal dander, bacteria, lead paint, household dust, cooking smoke and grease, wood and tobacco smoke, and smog.

11. A method according to claim 1, wherein the air-pressure control system further includes an adjustable frame and the window includes a window frame, the adjustable frame including a top rail and a first and second adjustable side rail, installing the air pressure control system within the window including:
inserting the air-pressure control system into the window such that the adjustable frame is within the window frame; and
adjusting the first and second side rails to expand the adjustable frame to fit the window frame.

12. A method according to claim 11, wherein the adjustable frame includes a tape measure having a zero point located at a center point of the top rail.

13. A method according to claim 12, wherein the tape measure further includes a first set of increasing numbers and a second set of increasing numbers, the first set of increasing numbers increasing to the left of the zero point and the second set of increasing numbers increasing the to the right of the zero point, adjusting the first and second side rails to expand the adjustable frame exposing the numbers from the first and second set of increasing numbers.

14. A method according to claim 13, wherein installing the air-pressure control system includes:
aligning the zero point with a center of the window frame; and
adjusting first and second side rails such that the exposed numbers from the first and second set of increasing numbers match.

15. A method according to claim 14, further comprising;
providing a first and second cover;
cutting the first and second covers based upon the exposed numbers from the first and second set of increasing numbers; and
installing the first and second covers into the adjustable frame to cover a first space between a side of the air-pressure control system and the first side rail and a second space between an opposing side of the air-pressure control system and the second side rail.

16. A method according to claim 1, wherein the air-pressure control system further includes:
a thermostat; and
a thermoelectric device configured to adjust the temperature of the air drawn into the system based upon a signal from the thermostat.

17. A method according to claim 1, wherein the air-pressure control system includes:
a heater located upstream of the germicidal radiation chamber; and
a thermocouple located within the germicidal radiation chamber, the thermocouple connected to the closed-loop controller and configured to measure a temperature of the air passing through the germicidal radiation chamber, the closed-loop controller adjusting power to the main heater based upon the measured temperature to heat the air passing through the air-pressure control system

18. A method according to claim 1, wherein the air-pressure control system further includes:
a humidistat located between the system inlet and the system outlet and configured to measure the humidity of the drawn air; and
a thermoelectric device connected to the humidistat and configured to dehumidify the drawn air based upon the measured humidity.

19. A system for reducing airborne contamination within a home comprising:
a housing defining the structure of the system and configured to fit within a window of the home;
an adjustable frame extending around at least part of the housing and configured to expand to at least one dimension of the window;
a system inlet;
a system outlet;
a variable-speed fan configured to operate at a speed;
a motor controller in communication with the fan and configured to control the speed of the fan;
a solid state anemometer configured to monitor an air pressure differential between the system inlet and the system outlet;
a closed-loop controller in communication with the motor controller and the solid state anemometer, wherein the closed-loop controller is configured to vary the speed of the fan based on the pressure differential between the inlet and outlet of the system; and
a germicidal radiation chamber located within an airflow path in the air-pressure control system, the germicidal radiation chamber including at least one UV light source and configured to sterilize the air as it passes through the airflow path, the sterilized air displacing an equal volume of contaminated air within the home.

20. A system according to claim 19, further comprising at least one filter located within the airflow path, the filter cleaning the drawn air by removing particulates from the drawn air as it passes through the filter.

21. A system according to claim 20, wherein the filter is located at a first end of the germicidal radiation chamber.

22. A system according to claim 21, further comprising a second filter located at a second end of the germicidal radiation chamber.

23. A system according to claim 19, wherein the system is configured to introduce the sterilized air into the building at a flowrate between 20 cubic feet per minute and 75 cubic feet per minute.

24. A system according to claim 19, wherein the system removes volatile organic compounds from the drawn air.

25. A system according to claim 19, wherein the air exiting the system contains substantially no particles between 5.0 microns and 0.3 microns.

26. A system according to claim 25, wherein the particles include at least one of dust mites, animal dander, bacteria, lead paint, household dust, cooking smoke and grease, wood and tobacco smoke, and smog.

27. A system according to claim 19, wherein the sterilized air displacing the contaminated air within the home improves the air quality within the home.

28. A system according to claim 19, wherein the contaminated air includes at least one of volatile organic compounds, airborne micro-contamination, harmful gases, dust mites, animal dander, bacteria, lead paint, household dust, cooking smoke and grease, wood and tobacco smoke, and smog.

29. A system according to claim 19, wherein the adjustable frame includes:
a top rail extending along a top surface of the housing;
a first adjustable side rail located on a first side of the housing; and
a second adjustable side rail located on a second side of the housing, the first and second adjustable rail configured to expand outwardly such that the adjustable frame fits a frame of the window.

30. A system according to claim 29, wherein the adjustable frame includes a tape measure having a zero point located at a center point of the top rail.

31. A system according to claim 30, wherein the tape measure further includes a first set of increasing numbers and a second set of increasing numbers, the first set of increasing numbers increasing to the left of the zero point and the second set of increasing numbers increasing the to the right of the zero point, the numbers from the first and second set of increasing numbers being exposed as the first and second rails are expanded outwardly.

32. A system according to claim 31, wherein the zero point is aligned with a center of the window frame when the system is installed in the window.

33. A system according to claim 31, wherein the adjustable frame further includes;
a first cover configured to cover a space between the first side of the housing and the first adjustable side rail; and
a second cover configured to cover a space between the second side of the housing and the second adjustable side rail.

34. A system according to claim 21, wherein the first and second covers are sized based on the exposed numbers on the tape measure.

35. A system according to claim 19, further comprising:
a thermostat; and
a thermoelectric device configured to adjust the temperature of the air drawn into the system based upon a signal from the thermostat.

36. A system according to claim 19, further comprising:
a heater located upstream of the germicidal radiation chamber; and
a thermocouple located within the germicidal radiation chamber, the thermocouple connected to the closed-loop controller and configured to measure a temperature of the air passing through the germicidal radiation chamber, the closed-loop controller adjusting power to the main heater based upon the measured temperature to heat the air passing through the air-pressure control system.

37. A system according to claim 1 further comprising:
a humidistat located between the system inlet and the system outlet and configured to measure the humidity of the drawn air; and
thermoelectric device connected to the humidistat and configured to dehumidify the drawn air based upon the measured humidity.

38. A system according to claim 19, wherein the airflow path is blackened to prevent UV reflection through the system inlet and system outlet.

39. A system according to claim 19, wherein the closed-loop controller includes:
a microprocessor configured to compare an output from the solid state anemometer and a setpoint value and adjust the speed of the fan based on the difference between the solid state anemometer output and the setpoint value.

40. A system according to claim 39, further comprising a safety sensor in communication with the microprocessor, the microprocessor configured to alarm when the system is not operating at the setpoint values.

41. A system according to claim 19, wherein the closed-loop controller is further configured to bring the fan to full speed upon a change in condition within the home, the closed-loop controller then reducing the speed of the fan to obtain a setpoint value.

42. A method for reducing airborne contamination within a home comprising: installing an air-pressure control system within a window of the home, the air-pressure control system including:
a system inlet,
a system outlet,
a variable-speed fan configured to operate at a speed,
a motor controller in communication with the fan and configured to control the speed of the fan,
a sensor configured to monitor an air pressure differential between the system inlet and the system outlet,
a closed-loop controller in communication with the motor controller and the sensor, wherein the closed-loop controller is configured to vary the speed of the fan based on the pressure differential between the inlet and outlet of the system, and
a germicidal radiation chamber located within an airflow path in the air-pressure control system, the germicidal radiation chamber including at least one UV light source;
drawing air from outside the home through the system inlet and the airflow path, the germicidal radiation chamber sterilizing the air as it passes through the airflow path; and
introducing the sterilized air into the home through the system outlet, the sterilized air displacing an equal volume of contaminated air within the home.

43. A method for reducing airborne contamination within a home comprising: installing an air-pressure control system within a window of the home, the air-pressure control system including:
a system inlet,
a system outlet,
a variable-speed fan configured to operate at a speed,
a motor controller in communication with the fan and configured to control the speed of the fan,
a solid state anemometer configured to monitor an air pressure differential between the system inlet and the system outlet, and
a closed-loop controller in communication with the motor controller and the solid state anemometer, wherein the closed-loop controller is configured to vary the speed of the fan based on the pressure differential between the inlet and outlet of the system;
drawing air from outside the home through the system inlet and the airflow path; and
introducing the drawn air into the home through the system outlet, the drawn air displacing an equal volume of contaminated air within the home.
